(19) United States
(12) Patent Application Publication
(10) Pub. No.: US 2007/0190631 A1
(43) Pub. Date: Aug. 16, 2007

(54) Catalyzed method for forming products from a liquid reactant

(76) Inventors: Michiel T. Kreutzer, Rotterdam (NL); Jacob A. Moulijn, Gravenhage (NL); Sten A. Wallin, Midland, MI (US); Freek Kapteijn, Tk Purmerend (NL); Karen M.de Lathouder, Gravenhage (NL)

Correspondence Address: THE DOW CHEMICAL COMPANY, INTELLECTUAL PROPERTY SECTION,, P. O. BOX 1967, MIDLAND, MI, 48641-1967, US

(21) Appl. No.: 10/589,996

(22) PCT Filed: Feb. 28, 2005
(86) PCT No.: PCT/US05/06347

§ 371 Date: Feb. 5, 2007

Related U.S. Application Data
(60) Provisional application No. 60/548,386, filed on Feb. 27, 2004.

(51) Int. Cl. C12P 1/00; C12N 9/00

(52) U.S. Cl. 435/183; 435/41

(57) ABSTRACT

A reaction product is made by flowing a liquid containing a reactant through a porous ceramic honeycomb wherein the porosity of the walls of the honeycomb are such that the liquid containing the reactant substantially penetrates into the walls and the reactant reacts as the liquid containing the reactant flows from the inlet end to the outlet end of the monolithic ceramic honeycomb.

### FIELD OF THE INVENTION

The invention relates to an improved method for forming products from a liquid reactant using a catalyst. In particular, the invention relates to a method of forming products using a bio-catalyst (e.g., enzymes).

### BACKGROUND OF THE INVENTION

Many commercially important reactions utilize a liquid reactant. Typical reactions utilizing a reactant in a liquid have employed a catalyst on a support. Numerous supports have been used such as silica, alumina, silica-alumina composites and carbon of various forms. Typically, these reactions have been carried out in reactors where the catalyst-supports are in the form of powders, beads or chips packed within the reactor (typically referred to as fixed bed reactors).

Unfortunately, these reactors have not proven useful for fast reactions because they tend to be diffusion limited. That is, if the diffusion is slow compared to the rate of reaction, only the outer shell of the catalyst structure is effectively used in the conversion of reactants that diffuse into the catalyst, while the inner part of the catalyst cannot be accessed by the reactants. As a result, not all the catalyst is used. In addition, diffusional limitations tend to reduce product quality such as lowered yield and presence of undesirable side reaction products. To overcome this diffusional limitation, the particle size of the catalyst-support may be reduced, but this increases the pressure drop through the reactor and causes other problems such as increased bed plugging.

Recently, in an attempt to improve upon the packed bed type reactors, ceramic honeycomb supports have been used. Even though these reactors have shown improved mass transfer and low pressure drop, they have seen limited application because the catalyst that can participate in the reaction as the liquid moves down the channel is limited to the small amount present on the walls of the channel. Further, the amount of catalyst is limited, because of this diffusion limitation and, for example, to plugging of the porosity with the catalyst, which causes catalyst in the wall to not participate in the reaction.

Accordingly, it would be desirable to provide a method for reacting a liquid or a reactant in a liquid that avoids one or more of the problems of the prior art such as those described above.

### SUMMARY OF THE INVENTION

The invention is a method for forming a reaction product from a liquid comprising;

(a) flowing a liquid containing a reactant into a ceramic honeycomb having an inlet end and outlet end connected by adjacent channels that extend from the inlet end to the outlet end of the ceramic honeycomb, the channels being defined by a plurality of interlaced thin porous partition walls having a catalyst thereon wherein the porosity of the partition walls are such that the liquid containing the reactant substantially penetrates into the walls and the reactant reacts as the liquid containing the reactant flows from the inlet end to the outlet end of the monolithic ceramic honeycomb and (b) collecting the reaction product from the outlet end of the ceramic honeycomb.

The method may be used in any application that may be useful to form a reaction product using a reactant in a liquid. Examples of reactions include hydrogenation, hydroxylation, enzyme catalyzed reactions, alkylation, oxidation, esterification, de-esterification and hydroformylation.

### DETAILED DESCRIPTION OF THE INVENTION

The method utilizes a ceramic honeycomb. The honeycomb may be of any useful shape and the channels of the honeycomb may have any useful shape (e.g., circular, square, rectangular and hexagonal). The ceramic honeycomb may be any suitable ceramic such as those known in the art. Exemplary ceramics include alumina, zirconia, silicon carbide, silicon nitride and aluminum nitride, silicon oxynitride and silicon carbonitride, mullite, cordierite, beta spodumene, aluminum titanate, strontium aluminum silicates, lithium aluminum silicates. Preferred ceramics include, silicon carbide, cordierite and mullite or combination thereof. Exemplary silicon carbides are described in U.S. Pat. Nos. U.S. Pat. No. 6,669,751B1 and WO publications EP 1142619A1, WO 2002/070106 A1. Other suitable porous bodies are described by WO 2004/011386A1, WO 2004/011124A1, US 2004/0020359 A1 and WO 2003/051488A1.

Preferably the ceramic honeycomb is a mullite honeycomb having an acicular microstructure. Examples of such acicular ceramics include those described by U.S. Pat. Nos. 5,194,154; 5,173,349; 5,198,007; 5,098,455; 5,340,516; 6,596,665 and 6,306,335; U.S. Patent Application Publication 2001/0038810; and International PCT publication WO 03/082773.

When the honeycomb is a ceramic having an acicular microstructure, the mean aspect ratio of the grains are generally greater than 2 (length twice as long as the width). Preferably the mean aspect ratio is at least about 10, more preferably at least about 15.

Several factors bear upon whether the liquid substantially penetrates into the walls of the honeycomb. Examples of such factors include the amount and type of porosity of the honeycomb and the surface tension. Generally, the liquid substantially penetrates when at least about 5% of the static liquid fraction is attributable to liquid that has penetrated the walls of the ceramic honeycomb. Preferably, at least about 10%, more preferably at least about 15%, even more preferably at least about 20%, and most preferably at least about 40% of the static liquid fraction is attributable to liquid that has penetrated the walls of the ceramic honeycomb as measured by a pulse dye residence time distribution (RTD) experiment under Taylor flow. Taylor flow is the alternating flow of bubbles and liquid through a channel as described by G. I. Taylor, Journal of Fluid Mechanics, 10, 161-165, (1960).

The percentage of liquid that penetrates is determined by subtracting the static liquid fraction of a layer that would be obtained for a dense honeycomb and liquid under the same conditions from the static liquid fraction of the method of the present invention. The static liquid fraction of a liquid flowing through a dense honeycomb may be calculated by known methods and correlations such as described by Aussilous et al, Physics of Fluids, 12(10), 2367-2371 (2001) and F. P. Bretherton, Journal of Fluid Mechanics, 10, 166-188, (1960).

It is understood that the method for determining the static liquid fraction involves flowing a liquid containing a reactant and a gas through the channels of the ceramic honeycomb. Generally, the gas-liquid flow has a capillary number of about 1×10⁻⁶ to about 0.1. Preferably the capillary number is at least about 1×10⁻⁵, more preferably at least about 1×10⁻⁴ to preferably at most about 0.05 and more preferably at most about 0.01. The capillary number is the flow velocity multiplied by the liquid viscosity divided by the surface tension of liquid-gas interphase, which is well known in the art and is described, for example, by Aussilous et al, Supra; F. P. Bretherton, Supra; Kolb, Chemical Engineering Science, 46 (9) 2181-2195, (1991); and Thulasidas, Chemical Engineering Science 50(2), 183-199, (1995).

The liquid substantially penetrates and reacts, is generally indicated by an increase in activity per gram of catalyst compared to a similar honeycomb having the same amount of catalyst located primarily on the channel walls (i.e., low porosity “<40%” honeycomb). This effect is typically more pronounced the faster the reaction is. Activity is the rate of conversion of reactant to product, for example, per unit monolith volume or per gram of catalytic species. Preferably, the activity per gram of catalyst increases by at least about 5%, more preferably at least about 10%, even more preferably 20%, and most preferably at least about 50% greater activity. In some cases the activity may increase by a factor of 2, 3 or 5 times.

The residence time distribution of liquid in the vessel is used to determine the stagnant liquid fraction herein. The residence time distribution is obtained by introducing a pulse of tracer at the feed of the reactor and recording when it leaves the reactor.

In determining the stagnant liquid fraction from the residence time distribution, the piston-dispersion-exchange (PDE) model is used. In this model, the fluid in the reactor is decomposed into a stagnant and a dynamic part. The dynamic part flows with an average velocity with random fluctuations superimposed on that average velocity. Further, matter can exchange between the dynamic and stagnant fraction. The model, when comparing theoretical curves with experimental curves, allows three parameters to be determined:
(a) the fraction of liquid in the vessel that may be regarded as stagnant (stagnant liquid fraction),
(b) The rate of exchange between the stagnant and dynamic liquid fraction (mass exchange), and
(c) The amplitude of the random velocity fluctuations (dispersion).
   The PDE model is well established and is described by Stegeman D, et al., Industrial & Engineering Chemistry Research, 35(2), 378-385 (1996) and Iliuta I, Thyrion F, et al., Chemical Engineering Science, 51, 4579-4593 (1996).

The particular porosity and pore size of the ceramic honeycomb walls that is suitable in the present invention is dependent on several factors such as the particular honeycomb, catalyst, liquid and the surface tension of the liquid. For the liquid containing the reactant to substantially penetrate the walls of the ceramic honeycomb, the porosity, generally, must be at least about 45%, preferably at least about 50%, more preferably at least about 55%. To maintain sufficient strength to operate, the porosity is generally at most about 85%, preferably at most about 80% and most preferably at most about 75%. In addition to the amount of porosity, the mean pore size, generally, needs to be at least about 3 micrometers in diameter. Preferably, the mean pore size is at least about 4 micrometers, more preferably at least about 5 micrometers, most preferably at least about 10 micrometers to preferably at most about 100 micrometers, more preferably at most about 50 micrometers, and most preferably at most about 25 micrometers in diameter.

The amount of pores and mean pore size may be determined using known micrographic techniques such as quantitative stereology of a scanning electron micrograph of a polished section using, for example, the intercept method described by Underwood in Quantitative Stereology, Addison-Wesley, Reading, Mass., (1970).

The catalyst may be surface-bound to at least a portion of the ceramic grains, incorporated into at least a portion of the ceramic grains or combinations thereof. The catalyst may be any suitable catalyst, such as those known in the art. In particular, the catalyst may be any one of the following preferred embodiments or combinations of them.

A first preferred catalyst is directly bound-metal catalysts, such as noble metals, base metals and combinations thereof. Examples of noble metal catalysts include platinum, rhodium, palladium, ruthenium, rhenium, silver and alloys thereof. Examples of base metal catalysts include copper, chromium, iron, cobalt, nickel, zinc, manganese, vanadium, titanium, scandium and combinations thereof. The metal catalyst, preferably, is in the form of a metal, but may be present as an inorganic compound, such as an oxide, nitride and carbide, or as a defect structure within the ceramic grains of the porous catalyst support. The metal may be applied by any suitable technique, such as those known in the art. For example, the metal catalyst may be applied by chemical vapor deposition.

A second preferred catalyst coating is one that is incorporated into the lattice structure of the ceramic grains of the porous catalyst support. For example, an element may be Ce, Zr, La, Mg, Ca, a metal element described in the previous paragraph or combinations thereof. These elements may be incorporated in any suitable manner, such as those known in the art and by methods described later.

A third preferred catalyst is a combination of ceramic particles having metal deposited thereon. These are typically referred to as wash coats. Generally, wash coats consist of micrometer sized ceramic particles, such as zeolite, aluminosilicate, silica, ceria, zirconia, barium oxide, barium carbonate and alumina particles that have metal deposited thereon. The metal may be any previously described for directly deposited metal. A particularly preferred wash coat catalyst coating is one comprised of alumina particles having a noble metal thereon. It is understood that the wash coat may be comprised of more than one metal oxide, such as alumina having oxides of at least one of zirconium, barium, lanthanum, magnesium and cerium.

A fourth preferred catalyst is a perovskite-type catalyst comprising a metal oxide composition, such as those described by Golden in U.S. Pat. No. 5,939,354.

A fifth preferred catalyst is one that is formed by and deposited on the catalyst support by calcining at a temperature of from about 300° C. to about 3000° C., a composition that comprises (a) an aqueous salt solution containing at least one metal salt and (b) an amphiphilic ethylene oxide containing copolymer, wherein the copolymer has an average molecular weight of greater than 400, an ethylene oxide content of 5 to 90 percent and an HLB of between −15 and 15, as described by Gruenbauer, et al., PCT Patent Application No. 99/18809. In addition, the catalyst may also be one such as described by U.S. Pat. No. 5,698,483 and PCT Patent Application No. 99/03627.

A sixth preferred catalyst is an enzyme or enzyme supported by wash coats previously described or high surface area carbon. The enzyme may also be supported by other suitable supports such as those known in the art. Preferred supports for the enzyme include carbon (e.g., carbon fibers), polyethyenimine, alginate gels, sol-gel coatings, or combination thereof. Preferably, the enzyme is a lipase, a lactase, a dehalogenase or combination thereof. More preferably the enzyme is a lipase, a lactase or combination thereof.

A seventh preferred catalyst is metal active site supported on a high surface area carbon (e.g. carbon fibers). Preferably, the metal active site is a hydrogenation catalyst such as Ru, Ni, Pt, Pd, etc.

An eighth preferred catalyst is an immobilized homogeneous catalysts (e.g. covalently attached metal compound). The immobilized catalytic molecule can be attached directly to the ceramic grains, to a washcoat or to high surface area carbon. Immobilization methods include those generally known to those skilled in the art such as attachment through silane coupling agents. (Examples of homogeneous catalysts that can be supported are metal Schiff base complexes, metal phosphine and diazaphosphacycle)

The amount of catalyst may be any suitable amount depending on the particular reaction. Generally, at least about 10 percent to essentially all of the ceramic grains are coated or contain a catalyst.

The liquid containing a reactant may be a liquid reactant or a reactant that has been dissolved in a liquid reactant. The liquid may have more than one reactant such as two liquid reactants mixed together or a solid reactant dissolved in a liquid reactant. Preferably, at least a portion of the liquid is a solvent. Any suitable solvent may be used depending on the reaction or reactions desired. Exemplary solvents include alcohols (e.g., ethanol, propanol and methanol), ethers (e.g., dimethylether, tetrahydrofuran), glycolethers (e.g. DOWANOL®), ketones (e.g., methylethyl ketone, acetone), dimethylsulfoxide, aliphatic hydrocarbons (e.g. hexane, heptane, octane, heptene), aromatic hydrocarbons (e.g. benzene, toluene), water or a combination thereof.

The liquid containing a reactant may be any practicable viscosity, but, generally, the viscosity is about 0.01 to about 1000 centipoise. Preferably the viscosity is at least about 0.1, more preferably at least about 0.25 and most preferably at least about 0.5 to preferably at most about 500, more preferably at most about 100, most preferably at most about 50 centipoises.

In a preferred embodiment of the invention, at least one of the reactants is a gas contained within the liquid containing a reactant. That is at least one of the reactants is a bubble flowing within the liquid containing a reactant. Preferably, such a reactant system is flowing under Taylor flow conditions previously described.

After the liquid containing the reactant flows through the ceramic honeycomb, the product of the reaction is collected by any suitable means such as those known in the art (e.g., distillation and filtration).

### EXAMPLES

The acicular mullite ceramic honeycombs employed in Illustrations 1 through 9 and in Examples 1 through 3 were prepared in a like manner to those described in WO 2003082773 A1.

Illustrations 1 through 23 and Comparative Illustrations 1 through 8

In illustrations 1 through 23 and Comparative Illustrations 1 through 8, 400 cells/inch² honeycombs, of about 4 cm in diameter, were stacked into a vertically mounted tube, such that by-pass of liquid between the tube wall and the sample was prevented and such that the open space between two samples was less than 2 mm. Water and air were distributed evenly over the entire cross section of the sample at the top of the column and forced at a set flow rate down through the column. A detectable tracer was injected into the liquid feed just above the column containing the sample. The tracer was injected in a period that was short compared to the residence time of liquid inside the column. Just below the last sample in the tube, the concentration of liquid leaving the vessel over time was recorded until no detectable tracer was found in the column effluent. The exit concentration versus time data was normalized using the area under the curve and the first moment of the curve to obtain a so-called E(theta) curve.

The E-curves were fitted to a PDE model, as described by Stegeman et al., Supra. to obtain the fraction of stagnant liquid inside the samples, and the rate of mass exchange between the stagnant liquid and the flowing liquid. The percent of the static liquid fraction penetrating the walls is calculated by subtracting the liquid film fraction calculated for a dense wall capillary according to page 75 and 76 of Hydrodynamics of Taylor Flow in Capillaries and Monolith Reactors, PH. D. Thesis, M. Kreutzer, Delft University Press, Netherlands, (2003) from the static liquid fraction obtained from the PDE fit, which is then divided by the static liquid fraction from the PDE fit and the value obtained is multiplied by 100.

For Illustrations 1-8, the honeycombs were acicular mullite having a wall porosity of about 60% and a mean pore diameter of about 9 micrometers. For Illustrations 9-15, the honeycombs were acicular mullite having a wall porosity of about 60% and a mean pore diameter of about 16 micrometers.

For illustrations 16-23, acicular mullite honeycombs having a wall porosity of about 60% and a mean pore diameter of approximately 16 micrometers were coated with colloidal silica by a dip coating procedure in a similar manner as described by Beauseigneur, et al., U.S. Pat. No. 5,334,570. Before dip coating, the acicular mullite honeycombs were dried at 393 K for 2 hours and cooled before immersion. The acicular mullite honeycombs were immersed into demineralized water diluted 15-wt % LUDOX SM-30 (ALDRICH, 7 nanometers colloidal silica particles with a specific surface area of 345 m²/g stabilized by sodium counter ions) solution for 2 minutes at constant immersion and withdrawal speeds of 0.5 mm/s. Subsequently, excess liquid was removed from the coated acicular mullite honeycombs by applying pressurized air for 1 minute on both sides. Next, the acicular mullite honeycombs were dried on a horizontal rotating device for 12 hours at ambient conditions. Finally, the coated acicular mullite honeycombs were calcined at 723 K for 4 hours.

This dip coating resulted in acicular mullite honeycombs with a silica washcoat of approximately 11-wt %. The silica washcoat completely covered the acicular mullite grains with a homogenous layer of approximately 0.6 micrometers. The specific surface area of the coated acicular mullite honeycombs was approximately 36 m²/gₜₒₜₐₗ (330 m²/g_{silica}).

For Comparative Illustrations 1-8, the honeycombs were cordierite having a wall porosity of about 35% and a mean pore diameter of about 3 micrometers. The stagnant liquid fraction, mass exchange and % liquid penetrated into the pores of these Illustrations and Comparative Illustrations appear in Table 1. In Table 2 the data from Table 1 was averaged and clearly shows that the % liquid penetrated is substantially greater than zero, even with a catalyst, whereas the comparative illustrations showed no such penetration.

TABLE 1

| | | Mean Pore | | liquid | gas | | Static | Liquid | Percent of |
|---|---|---|---|---|---|---|---|---|---|
| | Honeycomb | diameter | Porosity | flowrate | flowrate | mass | liquid | film | SLF* |
| Illustration | material | (μm) | (%) | (L/hr) | (L/hr) | exchange | fraction | fraction | penetrated |
| Comparative 1 | cordierite | 3 | 35 | 300 | 600 | 0.52 | 0.13 | 0.14 | −8% |
| Comparative 2 | cordierite | 3 | 35 | 500 | 600 | 0.53 | 0.16 | 0.16 | −3% |
| Comparative 3 | cordierite | 3 | 35 | 600 | 800 | 0.32 | 0.15 | 0.17 | −13% |
| Comparative 4 | cordierite | 3 | 35 | 600 | 800 | 0.40 | 0.16 | 0.16 | 0% |
| Comparative 5 | cordierite | 3 | 35 | 600 | 900 | 0.36 | 0.15 | 0.16 | −7% |
| Comparative 6 | cordierite | 3 | 35 | 600 | 900 | 0.29 | 0.15 | 0.16 | −7% |
| Comparative 7 | cordierite | 3 | 35 | 400 | 600 | 0.53 | 0.15 | 0.16 | −7% |
| Comparative 8 | cordierite | 3 | 35 | 750 | 600 | 0.29 | 0.14 | 0.16 | −14% |
| 1 | mullite | 9 | 60 | 600 | 900 | 0.50 | 0.26 | 0.16 | 38% |
| 2 | mullite | 9 | 60 | 200 | 600 | 0.67 | 0.28 | 0.12 | 57% |
| 3 | mullite | 9 | 60 | 500 | 600 | 0.40 | 0.30 | 0.16 | 45% |
| 4 | mullite | 9 | 60 | 600 | 600 | 0.42 | 0.27 | 0.18 | 33% |
| 5 | mullite | 9 | 60 | 700 | 600 | 0.44 | 0.24 | 0.19 | 21% |
| 6 | mullite | 9 | 60 | 700 | 600 | 0.43 | 0.23 | 0.19 | 17% |
| 7 | mullite | 9 | 60 | 800 | 600 | 0.42 | 0.25 | 0.20 | 20% |
| 8 | mullite | 9 | 60 | 800 | 600 | 0.37 | 0.24 | 0.20 | 17% |
| 9 | mullite | 16 | 60 | 600 | 900 | 1.16 | 0.22 | 0.16 | 27% |
| 10 | mullite | 16 | 60 | 200 | 600 | 1.68 | 0.23 | 0.12 | 48% |
| 11 | mullite | 16 | 60 | 400 | 600 | 1.77 | 0.24 | 0.15 | 38% |
| 12 | mullite | 16 | 60 | 600 | 600 | 0.91 | 0.22 | 0.18 | 18% |
| 13 | mullite | 16 | 60 | 600 | 600 | 1.02 | 0.22 | 0.18 | 18% |
| 14 | mullite | 16 | 60 | 750 | 600 | 0.90 | 0.22 | 0.20 | 9% |
| 15 | mullite | 16 | 60 | 750 | 600 | 0.89 | 0.21 | 0.20 | 5% |
| 16 | Coated | ND | 50# | 700 | 1000 | 0.96 | 0.23 | 0.17 | 26% |
| | mullite | | | | | | | | |
| 17 | Coated | ND | 50# | 600 | 600 | 1.03 | 0.21 | 0.17 | 19% |
| | mullite | | | | | | | | |
| 18 | Coated | ND | 50# | 500 | 600 | 1.17 | 0.21 | 0.16 | 24% |
| | mullite | | | | | | | | |
| 19 | Coated | ND | 50# | 700 | 600 | 0.88 | 0.22 | 0.19 | 14% |
| | mullite | | | | | | | | |
| 20 | Coated | ND | 50# | 700 | 400 | 0.93 | 0.23 | 0.22 | 4% |
| | mullite | | | | | | | | |
| 21 | Coated | ND | 50# | 800 | 600 | 1.08 | 0.21 | 0.20 | 5% |
| | mullite | | | | | | | | |
| 22 | Coated | ND | 50# | 500 | 600 | 1.21 | 0.24 | 0.16 | 32% |
| | mullite | | | | | | | | |
| 23 | Coated | ND | 50# | 500 | 600 | 1.19 | 0.22 | 0.16 | 25% |
| | mullite | | | | | | | | |

*SLF = static liquid fraction
negative values for the “percent of SLF penetrated” means that no liquid penetrated the walls.
ND = not determined, but is less than 16 due to coating of pore walls.
#calculated based upon coating of pore walls with silica catalyst coating.

TABLE 2

| | | | | | Average | Average | Average |
|---|---|---|---|---|---|---|---|
| | | Mean Pore | | Average | Static | Liquid | Percent of |
| | Honeycomb | diameter | Porosity | mass | liquid | film | SLF* |
| Illustrations | material | (μm) | (%) | exchange | fraction | fraction | penetrated |
| Comparative 1-8 | Cordierite | 3 | 35 | 0.41 | 0.15 | 0.16 | −7.23% |
| 1-8 | Mullite | 9 | 60 | 0.46 | 0.26 | 0.18 | 31.15% |
| 9-15 | Mullite | 16 | 60 | 1.19 | 0.22 | 0.17 | 23.26% |
| 16-23 | Coated mullite | ND | 50* | 1.06 | 0.22 | 0.18 | 18.60% |

ND = not determined, but is less than 16 due to coating of pore walls.
*calculated based upon coating of pore wall silica catalyst coating.

### Example 1

A 200 cell/inch² honeycomb (60% porosity) of acicular mullite 4.3 cm diameter×5 cm long was dipped for 5 minutes (min) at room temperature in a 65% sucrose solution in water. After impregnation, excess solution was removed from the channels by blowing air through the channels. The sample was dried under continuous rotation for 24 hours at room temperature, followed by drying at 393 Kelvin (K) for 3 hours. The sample was carbonized in a tubular quartz reactor placed in a horizontal furnace. The sample was heated at 10 K/min in a N₂ stream up to 823 K in a tubular quartz reactor. The sample was maintained at 823 K for 2 hours under 500 milliliter (mL)/min N₂ flow to prepare the carbonized support. After carbonization the support was 15 wt % carbon, as calculated by weight gain.

A lipase (Candida rugosa (E.C. 3.1.1.3, type VII), obtained from Sigma) was immobilized on the carbonized support by recirculating a 2 g/L, pH 7 solution of the enzyme through the monolith mounted in a glass tube with a peristaltic pump. After 5 hours, ca. 350 mg of lipase had been immobilized on the support. The sample was removed, washed with water and air dried. After subsequent rehydration in 0.4 milli-molar (mM) DMSO/tris buffer (1:9), pH 7, the monolith exhibited an enzymatic activity of 0.19 milli-moles (mmol)/L-min as measured by the hydrolysis of p-nitrophenyl propionate (pNPP). The hydrolysis of pNPP was followed spectrophotometrically at 348 nm.

### Example 2

Furfuryl alcohol (90 mL) and pyrrole (27 mL) were stirred at 293 K. Acid catalyst (7 mL, 65% HNO₃) was added stepwise over a period of 45 min. During this exothermic reaction, temperature was kept constant at 293 K by using an ice bath. Polymerization was continued for 1 hour at 293 K.

A 200 cell/inch² honeycomb (60% porosity) of acicular mullite 4.3 cm diameter×5 cm long was dip coated for 5 min in the partially polymerized mixture. Excess liquid was blown out with nitrogen and the dip coating was repeated after 5 min. The impregnated polymer was solidified for 4 hours at ambient conditions, and polymerization was continued overnight at 353 K. Carbonization of the polymer was performed in a quartz reactor at 823 K (10 K/min, 300 mL/min, 100% Ar) for 2 hours. After carbonization, the support was 30% carbon, as calculated by weight gain.

A lipase (Candida rugosa (E.C. 3.1.1.3, type VII), obtained from Sigma) was immobilized on the carbonized support by the same procedure described in Example 1. After 5 hours, ca. 190 mg of lipase had been immobilized on the support. After subsequent rehydration in 0.4 mM DMSO/tris buffer (1:9), pH 7, the monolith exhibited an enzymatic activity of 0.091 mmol/L-min as measured by the hydrolysis of pNPP.

### Example 3

A carbon nanofiber-based coating were prepared by the method described by Nijhuis et al., Chemical Engineering Science, 56(3), 823-829 (2001). 300 g of Puralox SBA 200 gamma-alumina (Condea) and 170 g of colloidal alumina (20% in water, Alfa Aesar) were mixed in 400 mL water. The pH was adjusted to 4.5 with nitric acid and the slurry ball-milled for 24 hours. The pH was again adjusted to 4.5 with nitric acid. A 200 cell/inch² honeycomb (60% porosity) of acicular mullite 4.3 cm diameter×5 cm sample was dipped in the slurry for 5 min and dried horizontally while slowly rotated. The sample was calcined at 723 K for 4 hours. Nickel was deposited on the support at 353 K from a 1 M aqueous urea solution. After reduction for 1 hour at 973 K, carbon nanofibers were grown in a quartz reactor following the method of Kovalenko, et al., Journal of Molecular Catalysis A: Chemical, 182-183, 73-80, (2002). The Ni impregnated sample was heated (10 K/min) in a N₂ stream to 973 K. The Ni was reduced for 1 hour in 20% H₂ in N₂. After cooling to 773-873 K carbon fibers were grown in propene or methane and H₂ in N₂ (total flow rate 200 mL/min). The support consisted of 6% carbon as determined by weight gain.

A lipase (Candida rugosa (E.C. 3.1.1.3, type VII), obtained from Sigma) was immobilized on the carbonized support by the same procedure described in Example 1. After 5 hours, ca. 1000 mg of lipase had been immobilized on the support. After subsequent rehydration in 0.4 mM DMSO/tris buffer (1:9), pH 7, the monolith exhibited an enzymatic activity of 0.25 mmol/L-min as measured by the hydrolysis of pNPP.

### Example 3

A 200 cell/inch² honeycomb (60% porosity) of acicular mullite 4.3 cm diameter×5 cm long was dipped in a 40% aqueous colloidal silica suspension (Ludox AS-40, Aldrich) for 5 min. Excess liquid was gently blown out of the channels and the sample dried in air overnight before calcination at 723 K for 4 hours. The sample was then soaked for 45 hours in a 10% (v/v) solution of N-[3-(trimethoxysilyl)propyl]polyethyleneimine hydrochloride (Aldrich) in 2-propanol. After soaking, the sample was washed two times with 2-propanol and two times with water.

A lactase (β-galactosidase from Aspergillus orzae (E.C. 3.1.2.23), obtained from Sigma) was immobilized on the carbonized support by recirculating a 2 g/L, pH 7 solution of the enzyme through the monolith mounted in a glass tube with a peristaltic pump. After 5 hours, ca. 350 mg of lactase had been immobilized on the support. The sample was removed and washed with water. The monolith exhibited an enzymatic activity of 0.015 mmol/L-min as measured by the hydrolysis of o-nitrophenyl-β-galactopyranoside (ONPG) in 50 mM tris buffer, pH 7. The hydrolysis of ONPG was followed spectrophotometrically at 405 nm.

What is claimed is:

1. A method of forming a reaction product from a liquid comprising:
(a) flowing a liquid containing a reactant into a ceramic honeycomb having an inlet end and outlet end connected by adjacent channels that extend from the inlet end to the outlet end of the ceramic honeycomb, the channels being defined by a plurality of interlaced thin porous partition walls having a catalyst thereon wherein the porosity of the partition walls are such that the liquid containing the reactant substantially penetrates into the walls and the reactant reacts as the liquid containing the reactant flows from the inlet end to the outlet end of the monolithic ceramic honeycomb; and
(b) collecting the reaction product from the outlet end of the ceramic honeycomb.

2. The method of claim 1, wherein the monolithic ceramic honeycomb has a porosity of at least about 50% and a mean pore size that is at least about 5 micrometers.

3. The method of claim 1, wherein the liquid containing the reactant penetrates in an amount that is at least 10% of a static liquid fraction as determined using the resident time distribution obtained under Taylor flow of a tracer pulsed into the liquid.

4. The method of claim 3 wherein the amount is at least about 15% of the static liquid fraction.

5. The method of claim 4, wherein the method has a mass exchange as calculated using an E-curve that is at least about 0.4.

6. The method of claim 5, wherein the mass exchange is at least about 0.7.

7. The method of claim 6, wherein the static liquid fraction is at least about 1.25.

8. The method of claim 1, wherein the catalyst is comprised of an enzyme.

9. The method of claim 1, wherein the catalyst is comprised of a precious metal, base metal or combination thereof.

10. The method of claim 1, wherein at least one of the reactants is introduced as a gas.

11. The method of claim 1, wherein at least one of the reactants is a gas in a bubble flowing concurrently with the liquid.

12. The method of claim 1, wherein the liquid is comprised of solvent having a dissolved reactant therein.

13. The method of claim 12, wherein the solvent is water.

14. The method of claim 1, wherein the monolithic ceramic honeycomb is an acicular ceramic.

15. The method of claim 1, wherein the acicular ceramic has a mean pore size of at least about 3 micrometers and an aspect ratio of at least about 2.

16. The method of claim 15, wherein the acicular ceramic has a mean pore size of at least about 5 micrometers.

17. The method of claim 15, wherein the acicular ceramic is acicular mullite.

18. The method of claim 1, wherein the catalyst is comprised of carbon.

19. The method of claim 18, wherein the carbon is carbon fibers.

20. The method of claim 19, wherein the catalyst is further comprised of an enzyme.
